(19) United States
(12) Patent Application Publication
(10) Pub. No.: US 2005/0070514 A1
(43) Pub. Date: Mar. 31, 2005

(54) Therapies for treating respiratory diseases

(76) Inventor: William Garth Rapeport, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/491,804

(22) PCT Filed: Oct. 3, 2002
(86) PCT No.: PCT/GB02/04542

(30) Foreign Application Priority Data
Oct. 5, 2001 (GB) 0123951.6

(51) Int. Cl. A61K 31/519; A61K 31/56

(52) U.S. Cl. 514/171; 514/252.16; 514/262.1

(57) ABSTRACT

This invention relates to treating respiratory diseases by administering a phosphodiesterase 4 inhibitor in combination with a β agonist and an anti-inflammatory steroid.

### AREA OF THE INVENTION

This invention relates to compositions and methods for preventing or reducing the onset of symptoms of a respiratory disease, or treating or reducing the severity of a respiratory disease. In particular it relates to compositions and methods for treating respiratory diseases by administering a phosphodiesterase 4 inhibitor (PDE4), a β adrenergic agonist (β agonist) and an anti-inflammatory corticosteroid (steroid), particularly one which is inhaled.

### BACKGROUND OF THE INVENTION

Identification of novel therapeutic agents for treating respiratory diseases is made difficult by the fact that multiple mediators are responsible for the development of a particular disease. Thus, for example in treating asthma, it seems unlikely that eliminating the effects of a single mediator will always have a substantial effect on all of the components of the disease. An alternative to the “mediator approach” is to regulate the activity of the cells responsible for the pathophysiology of the disease. Accordingly, it could be useful to combine therapies in light of the fact that the etiology of many respiratory diseases involves multiple mediators. In this invention there is presented the combination of a PDE 4 inhibitor, a long-acting β agonist and a steroid, for treating respiratory diseases.

### SUMMARY OF THE INVENTION

In a first aspect this invention relates to a method of prophylaxis of, treating, or reducing the exacerbations associated with a respiratory disease by administering to a patient in need thereof an effective amount of a PDE4 inhibitor, a β agonist, and an anti-inflammatory steroid either in a single combined form, separately, or separately and sequentially where the sequential administration is close in time, or remote in time.

In a second aspect this invention relates to a composition for the prophylaxis of, treating, or reducing the exacerbations associated with a respiratory disease comprising an effective amount of a PDE4 inhibitor, an effective amount of β agonist, an effective amount of an anti-inflammatory steroid and a pharmaceutically acceptable excipient.

In a third aspect this invention relates to a method for preparing a composition which is effective for the prophylaxis of, treating, or reducing the exacerbations associated with, a respiratory disease which method comprises mixing an effective amount of a PDE4 inhibitor, an effective amount of a β agonist, and an effective amount of an anti-inflammatory steroid with a pharmaceutically acceptable excipient.

In a fourth aspect there is provided use of an effective amount of a PDE4 inhibitor, a β agonist, and an anti-inflammatory steroid in the manufacture of a medicament or medicament pack for the prophylaxis of, treating, or reducing the exacerbations associated with a respiratory disease.

In a fifth aspect there is provided use of a composition comprising an effective amount of a PDE4 inhibitor, a β agonist, an anti-inflammatory steroid and a pharmaceutically acceptable excipient in the manufacture of a medicament for the prophylaxis of, treating, or reducing the exacerbations associated with a respiratory disease.

### DETAILED DESCRIPTION OF THE INVENTION

The combination therapy contemplated by this invention comprises administering a PDE4 inhibitor, a β agonist and a steroid to prevent onset of a disease of the respiratory system or to treat an existing condition. The actives may be administered together in individual, binary or triple dosage forms. Or they may be administered as different formulations. They may be administered at the same time. Or they may be administered either close in time or remotely, such as where one or two active(s) is/are administered in the morning and the other active(s) is/are administered in the evening. Thus for example a β-agonist and steroid may be formulated together in a binary dosage form and a PDE4 inhibitor as an individual dosage form, and the two dosage forms may be administered simultaneously, separately or sequentially. The combination may be used prophylactically or after the onset of symptoms has occurred. In some instances the combination may be used to prevent the progression of a respiratory disease or to arrest the decline of a respiratory function such as lung function.

The combination of the three actives described herein can be used to treat diseases of the respiratory system, in particular allergic and inflammatory diseases of the lungs and upper respiratory tract. Exemplary diseases include: asthmatic conditions (e.g., allergic asthma, bronchial asthma, exercise-induced asthma, pollution-induced asthma (PIA) and cold-air induced asthma), cough, chronic obstructive pulmonary disease (COPD), different conditions of bronchitis (e.g., acute bronchitis, chronic bronchitis, obstructive bronchitis, spastic bronchitis, allergic bronchitis), and rhinitis (e.g., seasonal or perennial rhinitis).

The preferred PDE4 inhibitor useful in this invention is any compound that inhibits the PDE4 enzyme primarily or exclusively. Compounds which inhibit other members of the PDE family as well as PDE4 are excluded, except for certain compounds which can be designated as mixed PDE3/PDE4 inhibitors. Generally it is preferred to use a compound which has an IC₅₀ ratio of about 0.1 or greater as regards the IC₅₀ for the PDE IV catalytic form which binds rolipram with a high affinity divided by the IC₅₀ for the form which binds rolipram with a low affinity.

PDE inhibitors used in treating inflammation and as bronchodilators, drugs like theophylline and pentoxyfyllin, inhibit PDE isozymes indiscriminately in all tissues. These compounds exhibit side effects, apparently because they non-selectively inhibit many or all PDE isozyme classes in all tissues. The targeted disease state may be effectively treated by such compounds, but unwanted secondary effects may be exhibited which, if they could be avoided or minimized, would increase the overall therapeutic effect of this approach to treating certain disease states. For example, clinical studies with the selective PDE4 inhibitor rolipram, which was being developed as an antidepressant, indicate it has psychotropic activity and produces gastrointestinal effects, e.g., pyrosis, nausea and emesis.

It turns out that there are at least two binding forms on human monocyte recombinant PDE4 (hPDE 4) at which inhibitors bind. One explanation for these observations is that hPDE 4 exists in two distinct forms. One binds the likes of rolipram and denbufylline with a high affinity while the other binds these compounds with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

A method for determining IC₅₀s ratios is set out in U.S. Pat. No. 5,998,428 which is incorporated herein in full by reference as though set out herein. See also PCT application WO 00/51599 for an another description of said assay. Compounds which demonstrate a ratio of 0.1 or greater as determined by the method described in that patent are within the scope of this invention.

Exemplary PDE inhibitors for the uses noted herein are:

Compounds set out in U.S. Pat. No. 5,552,438, in particular cis-4-cyano-4-[3-(cyclopentyloxy)-4-methoxyphenyl]cyclohexane-1-carboxylic acid. Its salts, esters, pro-drugs or physical forms are also preferred. Its USAN name is cilomilast. It is also identified by the marks Ariflo® and Zariflo.

A 9-benzyladenine derivative nominated NCS-613 (INSERM).

D-4418 from Chiroscience and Schering-Plough.

K-34 from Kyowa Hakko.

AWD 12-281 from ASTA Medica (Hofgen, N. et al. 15th EFMC Int Symp Med Chem (Sep. 6-10, Edinburgh) 1998, Abst P.98; CAS reference No. 247584020-9).

A benzodiazepine identified as Cl-1018 (PD-168787 and attributed to Pfizer.

A benzodioxole derivative disclosed by Kyowa Hakko in WO 9916766.

V-11294A made by Napp (Landells, L.J. et al. Eur Resp J [Annu Cong Eur Resp Soc (Sep. 19-23, Geneva) 1998] 1998, 12(Suppl. 28): Abst P2393).

Roflumilast (CAS reference No 162401-32-3); or pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide. The latter is a mixed PDE3/PDE4 inhibitor. Both have been prepared and published on by Byk-Gulden, now Altana.

T-440 (Tanabe Seiyaku; Fuji, K. et al. J Pharmacol Exp Ther, 1998, 284(1): 162) and T2585.

Arofylline under development by Almirall-Prodesfarma.

VM554/UM565 from Vemalis.
(R)-(+)-1-(4-bromobenzyl)-4-[(3-cyclopentyloxy)-4-methoxyphenyl]-2-pyrrolidone;
(R)-(+)-1-(4-bromobenzyl)-4-[(3-cyclopentyloxy)-4-methoxyphenyl]-2-pyrrolidone;
3-(cyclopentyloxy-4-methoxyphenyl)-1-(4-N′-[N2-cyano-S-methyl-isothioureido]benzyl)-2-pyrrolidone;
cis 4-cyano-4-(3-cyclopentyloxy-4-methoxyphenyl)cyclohexan-1-carboxylic acid];
cis-[4-cyano-4-(3-cyclopropylmethoxy-4-difluoromethoxyphenyl)cyclohexan-1-ol];
(R)-(+)-ethyl[4-(3-cyclopentyloxy-4-methoxyphenyl)pyrrolidine-2-ylidene]acetate; and
(S)-(−)-ethyl[4-(3-cyclopentyloxy-4-methoxyphenyl)pyrrolidine-2-ylidene]acetate.

Most preferred are those PDE4 inhibitors which have an IC₅₀ ratio of greater than 0.5, and particularly those compounds having a ratio of greater than 1.0. Preferred compounds are cis4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl) cyclohexan-1-one and cis-[4-cyano-4-(3-cyclopropylmethoxy-4-difluoromethoxyphenyl)cyclohexan-1-ol]; these are examples of compounds which bind preferentially to the low affinity binding site and which have an IC₅₀ ratio of 0.1 or greater.

Other possible PDE-4 and mixed PDE3/PDE4 inhibitors include those listed in WO01/13953, the disclosure of which is hereby incorporated by reference.

The β agonists used in this invention will be any compound, which has been, is, or may be used to treat a respiratory disease. Its scope includes non-selective β agonists such as epinephrine, norepinephrine, colterol, ethyinorepinephrine, isoproterenol, metaproterenol, ephedrine and selective β₂ agonists. No distinction is made between orally administered compounds or those administered via an inhaled or intranasal formulation.

The selective β₂-agonists are preferred, particularly those routinely administered by inhaled formulations. Most preferred are those β₂-agonists which have a long-lasting effect, by which is meant that the drug will have an effect on the bronchi that lasts around 6 hours or more, up to 12 hours in some instances.

Exemplary β₂ agonists include, for example: metaproterenol, terbutaline (e.g. as sulfate), albuterol (e.g. as free base or as sulfate), salmefamol, isoetharine, pirbuterol (e.g. as acetate), bitolterol, fenoterol (e.g. as hydrobromide), formoterol (e.g. as fumarate), procaterol, salmeterol (e.g. as xinafoate), ritodrine, AR-C68397AA, broxaterol, CHF-1035, HOKU-81, ibuterol, KUL-1248, soterenot, meluadrine, TA-2005, tiaramide, levosalbutamol, tulobuterol, carbuterol, reproterol (e.g. as hydrochloride), clenbuterol, hexoprenaline, orciprenaline, isoprenaline, rimiterol, procaterol, bambuterol, biolterol or mabuterol. See also the β₂ agonists disclosed in UK application GB 0103630.0 filed 14 Feb. 2001. The more preferred compounds are salmeterol (in particular the xinafoate), and formoterol (in particular the fumarate). The salts, esters, solvates, and polymorphs of these compounds are included within the scope of this invention. Salmeterol in particular is preferred. These compounds are either commercially available or have been described in the scientific literature.

The steroids which are useful in this invention are those oral and inhaled corticosteroids and their pro-drugs which have anti-inflammatory activity. Examples are methyl prednisolone, prednisone, dexamethasone, fluticasone and its esters (e.g. the propionate ester) 6α, 9α-difluoro-17α-[(2-furanylcarbonyl)oxy]-11β-hydroxy-16α-methyl-3-oxo-androsta-1,4-diene-17β-carbothioic acid S-fluoromethyl ester), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone (e.g. as the 17-propionate ester or the 17,21-dipropionate ester, budesonide, flunisolide, mometasone (e.g. as the furoate ester), triamcinolone (e.g as the acetonide) rofleponide, ciclesonide, butixocort propionate, RPR-106541 and 5T-126 (SSP-Torii).

Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl) oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Methyl prednisolone and prednisone are oral and injectable forms of anti-inflammatory corticosteroids; they are available from numerous branded and generic pharmaceutical companies. Beclomethasone dipropionate is sold as an aerosol for inhalation under the names Beconase® and Beconase AQ® by GlaxoSmithKline. Fluticasone propionate is sold under the name Flonase® by GlaxoSmithKline. Triamcinolone acetonide is sold by Rhone-Poulenc Roher under the name Nasacort® as a nasal spray and aerosol. Flunisolide is sold as a nasal solution under the name Nasalide® and Nasarel™ by Roche Laboratories. Dexamethasone is sold as the sodium phosphate salt by Medeva Pharmaceuticals, Inc. under the name Dexacort™ Phosphate. Mometasone furoate is sold as the monohydrate as a nasal preparation by Schering Corp under the name Nasonex®. Budesonide is yet another inhaled corticosteriod used in treating pulmonary diseases. It is market by Astra Pharmaceuticals, L.P. as a powder in a Turbuhaler® device under the name Pulmicort Turbuhaler®. All of these drugs and nasal preparations or oral or injectable formulations can be found in the 1999 edition of the Physicians' Desk Reference® (PDR), published by Medical Economics Corporation, Inc, of New Jersey, USA.

Exemplary preferred combinations are: i) cilomilast, formoterol and budesonide; ii) roflumilast, formoterol and budesonide; iii) cilomilast, fluticasone propionate and salmeterol; iv) roflumilast, fluticasone propionate and salmeterol; v) AWD 12-281, formoterol and budesonide; vi) AWD 12-281, fluticasone propionate and salmeterol, viii) cilomilast, formoterol and fluticasone propionate; viii) roflumilast, formoterol and fluticasone propionate; and ix) AWD 12-281, formoterol and fluticasone propionate.

These drugs are usually administered as an oral preparation or a nasal spray or aerosol, or as an inhaled powder. This invention contemplates co-administering two or three of the actives in one delivery form such as an inhaler, that is, putting two or three actives in the same inhaler.

The present compounds and pharmaceutically acceptable salts which are active when given orally can be formulated as syrups, tablets, capsules, controlled-release preparation or lozenges or as an inhalable preparation.

The most suitable route of administration may depend upon factors such as the nature of the condition or disorder to be treated.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A syrup formulation will generally consist of a suspension or solution of the compound or salt in a liquid carrier for example, ethanol, peanut oil, olive oil, glycerine or water with a flavoring or coloring agent.

Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of each therapeutically active ingredient. Alternatively, the active ingredient(s) may be presented without excipients. Packaging of the formulation may be suitable for unit dose or multi-dose delivery. In the case of multi-dose delivery, the formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing one or more actives, preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the active ingredient(s) and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants eg oleic acid or lecithin and cosolvents eg ethanol. Pressurised formulations will generally be retained in a canister (eg an aluminium canister) closed with a valve (eg a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means eg by micronisation. The desired fraction may be separated out by air classification or sieving. Preferably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

Pressurized aerosol compositions will generally be filled into canisters fitted with a valve, especially a metering valve. Canisters may optionally be coated with a plastics material e.g. a fluorocarbon polymer as described in W096/32150. Canisters will be fitted into an actuator adapted for buccal delivery.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

Each dosage unit for oral administration contains suitably from 0.3 mg to 60 mg/Kg, and preferably from 1 mg to 30 mg/Kg of a compound or a pharmaceutically acceptable salt thereof. Preferred doses include 1 mg and 60 mg/Kg for treating COPD. Each dosage unit for parenteral administration contains suitably from 0.1 mg to 100 mg/Kg, of the compound or a pharmaceutically acceptable salt thereof. Each dosage unit for intranasal administration contains suitably 1-400 mcg and preferably 10 to 200 mcg per activation. A dry powder inhalation dose could contain 1-1000 micrograms per dose unit. A topical formulation contains suitably 0.001 to 5.0% of a present compound.

The amount of each active which is required to achieve a therapeutic effect will, of course, vary with the particular active, the route of administration, the subject under treatment, and the particular disorder or disease being treated.

The active ingredients may be administered from 1 to 6 times a day, sufficient to exhibit the desired activity. Preferably, the active ingredients are administered once or twice a day.

It is contemplated that all three active agents would be administered at the same time, or very close in time. Alternatively, one or two actives could be taken in the morning and the other(s) later in the day. Or in another scenario, one or two actives could be taken twice daily and the other(s) once daily, either at the same time as one of the twice-a-day dosing occurred, or separately. Preferably at least two, and more preferably all three, of the actives would be taken together at the same time. Preferably, at least two, and more preferably all three, actives would be administered as an admixture.

The following examples are provided to illustrate how to make and use the invention. They are not in any way intended to limit the scope of the invention in any manner or to any degree. Please refer to the claims for what is reserved to the inventors hereunder.

### EXAMPLES

PDE 4 Versus Rolipram High Affinity Binding

### Example 1

### Phosphodiesterase and Rolipram Binding Assays

### Example 1A

Isolated human monocyte PDE 4 and hrPDE (human recombinant PDE4) is determined to exist primarily in the low affinity form. Hence, the activity of test compounds against the low affinity form of PDE 4 can be assessed using standard assays for PDE 4 catalytic activity employing 1 μM [³H]cAMP as a substrate (Torphy et al., J. of Biol. Chem., Vol. 267, No. 3 pp1798-1804, 1992).

Rat brain high speed supematants were used as a source of protein. Enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, and 1 nanoM of [³H]-rolipram (Torphy et al., J. of Biol. Chem., Vol. 267, No. 3 pp1798-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP and [³H]5′-AMP were not present.

### Example 1B

Measurement of Phosphodiesterase Activity

PDE activity is assayed using a [³H]cAMP scintillation proximity assay (SPA) or [³H]cGMP SPA enzyme assay as described by the supplier (Amersham Life Sciences). The reactions were conducted in 96-well plates at room temperature, in 0.1 ml of reaction buffer containing (final concentrations): 50 mM Tris-HCl, pH 7.5, 8.3 mM MgCl2, 1.7 mM EGTA, [³H]cAMP or [³H]cGMP (approximately 2000 dpm/pmol), enzyme and various concentrations of the inhibitors. The assay was allowed to proceed for 1 hr and was terminated by adding 50 μl of SPA yttrium silicate beads in the presence of zinc sulfate. The plates were shaken and allowed to stand at room temperature for 20 min. Radiolabeled product formation was assessed by scintillation spectrometry. Activities of PDE3 and PDE7 were assessed using 0.05 μM [³H]cAMP, whereas PDE4 was assessed using 1 μM [³H]cAMP as a substrate. Activity of PDE1B, PDE1C, PDE2 and PDE5 activities were assessed using 1 μM [³H]cGMP as a substrate.

[³H]R-Rolipram Binding Assay

The [³H]R-rolipram binding assay was performed by modification of the method of 30 Schneider and co-workers, see Nicholson, et al., Trends Pharmacol. Sci., Vol. 12, pp. 19-27 (1991) and McHale et al., Mol. Pharmacol., Vol. 39, 109-113 (1991). R-rolipram binds to the catalytic site of PDE4 see Torphy et al., Mol. Pharmacol., Vol. 39, pp. 376-384 (1991). Consequently, competition for [³H]R-rolipram binding provides an independent confirmation of the PDE4 inhibitor potencies of unlabeled competitors. The assay was performed at 30° C. for 1 hr in 0.5 μl buffer containing (final concentrations): 50 mM Tris-HCl, pH 7.5, 5 mM MgCl₂, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/B filters that had been soaked in 0.3% polyethylenimine. The filters were washed with an additional 7.5-ml of cold buffer, dried, and counted via liquid scintillation spectrometry.

The foregoing statements and examples are intended to illustrate the invention, not to limit it. Reference is made to the claims for what is reserved to the inventors hereunder.

### Formulation Examples

A: Metered Dose Inhalers

TABLE 2

| | Per actuation | |
|---|---|---|
| Cilomilast | 18 | mcg |
| Fluticasone propionate | 50 or 100 or 250 | mcg |
| Salmeterol xinafoate | 36.25 | mcg |
| 1,1,1,2-Tetrafluoroethane | to 75.0 | mg |

The micronised active ingredients (e.g. for 120 actuations) are weighed into an aluminum can, 1,1,1,2-tetrafluoroethane is then added from a vacuum flask and a metering valve is crimped into place.

B: Dry Powder Inhalers

TABLE 3

| | Per cartridge or blister | |
|---|---|---|
| Cilomilast | 15 | mcg |
| Fluticasone propionate | 100 or 250 or 500 | mcg |
| Salmeterol xinafoate | 72.5 | mcg |
| Lactose Ph. Eur. | to 12.5 or 25 | mg |

The active ingredients are micronised and bulk blended with the lactose in the proportions given above. The blend is filled into hard gelatin capsules or cartridges or in specifically constructed double foil blister packs to be administered by an inhaler such as a Rotahaler, Diskhaler, or Diskus inhaler (each of these being a trademark of Glaxo Group Limited).

C. Formulation for Nasal Administration

TABLE 4

| Cilomilast | 150 | mg |
|---|---|---|
| Fluticasone propionate | .05 | g |
| Phenylethyl alcohol | 0.25 | mL |
| Microcrystalline cellulose | 1.5 | mg |
| and carboxymethylcellulose sodium (Avicel RC591) | | |
| Benzalkonium chloride | 0.02 | mg |
| Hydrochloric acid | to pH 5.5 | |
| Purified water | to 100 | mL. |

In a 100 μl metered volume dispensed by a Valois VP7 pre-compression pump, approximately 15 mcg of cilomilast and 10 mcg of tiopropium will be delivered.

D. Oral Tablet/Inhaled Formulation

The following tables 5, 6 and 7 illustrate a combination therapy where a tablet is used to deliver the orally available PDE inhibitor and a dry powder or metered dose inhaler is used for delivering the β₂-agonist and steroid. A treatment regimen would involve taking the tablet containing the PDE inhibitor once or twice a day and, in parallel or at a different time, taking the β₂-agonist and steroid, twice per day; or as prescribed and needed.

Table 5 sets out a tablet formulation which can be used to administer a PDE3/PDE4 inhibitor.

TABLE 5

| Composition | Unit Formula |
|---|---|
| Cilomilast | 15.0 mg |
| Lactose, Monohydrate | 99.64 mg |
| Microcrystalline Cellulose | 70.0 mg |
| Sodium Starch Glycolate | 10.0 mg |
| Magnesium Stearate | 2.0 mg |
| Total weight | 200.0 mg |

Tablet preparation is by conventional means using standard dry-powder mixing and a compression tableting tool.

Dry Powder Inhaler

Table 6 provides a formulation for β₂-agonist and steroid which can be administered in parallel, time-wise, with the PDE-containing tablet, or separately in time.

TABLE 6

| | Per cartridge or blister | |
|---|---|---|
| Fluticasone propionate | 100 or 250 or 500 | mcg |
| Salmeterol xinafoate | 72.5 | mcg |
| Lactose Ph. Eur. | to 12.5 or 25 | mg |

The active ingredients are micronised and bulk blended with the lactose in the proportions given above. The blend is filled into hard gelatin capsules or cartridges or in specifically constructed double foil blister packs to be administered by an inhaler such as a Rotahaler, Diskhaler, or Diskus inhaler (each of these being a trademark of Glaxo Group Limited).

Metered Dose Inhaler

Table 7 provides a formulation for β₂-agonist and steroid which can be administered in parallel, time-wise, with the PDE-containing tablet, or separately in time.

TABLE 7

| | Per actuation | |
|---|---|---|
| Fluticasone propionate | 50 or 100 or 250 | mcg |
| Salmeterol xinafoate | 36.25 | mcg |
| 1,1,1,2-Tetrafluoroethane | to 75.0 | mg |

The micronised active ingredients (e.g. for 120 actuations) are weighed into an aluminum can, 1,1,1,2-tetrafluoroethane is then added from a vacuum flask and a metering valve is crimped into place.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1-3. (canceled).

4. A method of prophylaxis of, treating, or reducing the exacerbations associated with a respiratory disease by administering to a patient in need thereof an effective amount of a PDE4 inhibitor, a β agonist, and an anti-inflammatory steroid either in a single combined form, separately, or separately and sequentially where the sequential administration is close in time, or remote in time.

5. A composition for the prophylaxis of, treating, or reducing the exacerbations associated with a respiratory disease comprising an effective amount of a PDE4 inhibitor, an effective amount of β agonist, an effective amount of an anti-inflammatory steroid and a pharmaceutically acceptable excipient.

6. A method for preparing a composition which is effective for the prophylaxis of, treating, or reducing the exacerbations associated with, a respiratory disease which method comprises mixing an effective amount of a PDE4 inhibitor, an effective amount of a β agonist, and an effective amount of an anti-inflammatory steroid with a pharmaceutically acceptable excipient.

7-9 (Delete)
